(19) United States
(12) Patent Application Publication
(10) Pub. No.: US 2024/0008949 A1
(43) Pub. Date: Jan. 11, 2024

(54) SYSTEM FOR MONITORING THE ORIENTATION OF MEDICAL DEVICES

(71) Applicant: GRAYSCALELAB KFT., Budapest (HU)

(72) Inventors: Péter GALAMBOS, Herceghalom (HU); Dénes Balázs HORVÁTHY, Tata (HU); József KUTI, Tura (HU); Péter SZÁNTÓ, Kaposvár (HU)

(73) Assignee: GRAYSCALELAB KFT., Budapest (HU)

(21) Appl. No.: 18/020,748

(22) PCT Filed: Sep. 9, 2021
(86) PCT No.: PCT/HU2021/050048

§ 371 Date: Feb. 10, 2023

(30) Foreign Application Priority Data
Aug. 19, 2020 (HU) P2000276

(51) Int. Cl. A61B 90/00

(52) U.S. Cl. CPC A61B 90/08; A61B 2090/0807

(57) ABSTRACT

System for monitoring the orientation of medical devices (1), which contains a medical device (1), a measuring device (4), a calibrating device (5) and an external device that communicates with the measuring device (4), which external device contains a communication module, a control unit, a program, a power source and display, characterised by that the measuring device (4) is connected to the medical device (1), which measuring device (4) contains a sensing system, which is preferably a gyroscope and accelerometer, a power source, a communication module and control unit, and the calibrating device (5) has at least one position adapted for calibrating the medical device (1).

### THE FIELD OF THE INVENTION

The object of the present invention relates to a system for monitoring the orientation of medical devices using an accelerometer and gyroscope (MEMS IMU sensor) in such a way that by performing calibration before measurement the relationship between the measured magnitude and the direction of the medical device in the world coordinate system are determined.

### THE STATE OF THE ART

Minimally invasive medical procedures performed with medical devices with the assistance of an imaging device are extremely widespread and frequently performed procedures.

Typically, in the course of imaging-guided invasive procedures an imaging device is used to map the body, the point on the skin where the medical device punctures the skin, the puncturing angle, and the puncturing depth at which it is necessary to perform the tissue sampling, ablation, or effusion drainage. Following this, the procedure is performed with the continual utilisation of the imaging device or with it being regularly switched on for the purpose of monitoring, through which the position of the medical device may be monitored. This is required because the deviation of the puncturing angle from that planned is frequently not obvious immediately after puncturing of the skin, and this only becomes apparent at a greater depth, which then has to be corrected. If correction is required it may become necessary to withdraw the medical device from the body and perform the puncturing once again, if the angle deviation is of such an extent that angle correction performed within the body is not possible.

Such a medical procedure is, for example, ablation, when the medical device is inserted into the tissue of a tumour in order to destroy the tumour cells with microwaves or radio waves emitted by the medical device, by freezing using the medical device, or by the injection of a chemical agent from the medical device.

Other such medical procedures include draining, in the case of which fluid is sucked out of or released from the body through the passage opened by the medical device.

Another such medical procedure is a biopsy, during which a sample is taken of tissue from the body of a human or an animal for the purpose of examining the removed sample afterwards under a microscope or using another method.

In the case that the target in the medical procedures mentioned above cannot be seen with the naked eye, it is necessary to monitor the path of the medical device in one way or another.

Due to the monitoring mentioned above the medical procedures may be combined with various imaging methods. For example, in the case of ultrasound-guided procedures, the movement of the medical device is constantly observed with the use of the ultrasound imaging device. The advantage of this is that the movement of the medical device may be tracked in real time, however deeper tissues and the organs within the skull and the chest cannot be visualised with ultrasound.

In those cases when the movement of the medical device within the body is monitored using tomographic procedures (CT and MRI), the body is irradiated at certain intervals or is placed in the path of the radiation emitted by the device in order to monitor the progress of the medical device. The reason for this is that the use of a CT scanner involves x-ray radiation, which may be damaging for the patient and the operation personnel. Also, in the case of MRI-guided procedures, the patient is not physically accessible for the doctor when the imaging is being performed in the MRI scanner; in addition the intense magnetic field created by the MRI scanner limits the range of devices that may be used.

In the course of tomographic procedures it is preferable to minimise the duration of the intervention in order to, among other things, reduce the x-ray load (in the case of a CT scan), the amount of energy used by the device, its depreciation, and to reduce the burden on the operation personnel.

It is worth using a device or system to monitor the orientation of the medical device so that the use of the tomographic imaging device is only required before starting the procedure in order to identify the target and, optionally, at the end of the procedure in order to check whether the target tissue has been reached with the medical device. With this the use of the tomographic imaging device may be limited to a minimum during the procedure. With such a device the duration of the intervention may be shortened, the duration of irradiation may be reduced in the case of certain tomographic imaging procedures and the number of angle corrections may also be reduced. In this way the duration of the entire intervention is shorter and the chance of the development of complications may also be reduced due to the lower number of angle corrections.

Naturally, the use of such a system may also be useful in the case of imaging procedures, such as ultrasound and X-ray imaging.

A possible solution for this is to monitor the position and direction of the medical device. Typical devices include devices provided with an accelerometer, such as that presented in the article of Wilkmann, C., Ito, N., Penzkofer, T. et al. Int J CARS (2015) 10: 629. Here an instrument provided with an accelerometer is used, which may be used in the case of a CT-guided biopsy. A MEMS IMU sensor, provided with an accelerometer, is placed on the medical device. This is used to determine the orientation of the medical device. It is a simple and cheap method, but it is unable to handle the rotation of the medical device around its own axis. This disadvantage does not make the device suitable for use in the course of everyday medical procedures.

USA patent publication document number US20180110569A1 presents a more complex device, which uses an accelerometer, gyroscope and a magnetometer. According to the publication document the magnetometer detects the position of the magnetic field of the Earth and determines at least one direction as compared to it, thereby making calibration unnecessary. The problem with this approach is that the determination of direction compared to the position of the Earth's magnetic field is interfered with by the iron content of nearby objects, such as reinforced concrete elements and certain medical apparatuses, furthermore a procedure using this does not provide precise calibration to a plane, in other words this approach may not be used efficiently in practice.

Furthermore, the device is unable to take into consideration rotation around its own axis; due to this the rotation of the device around its own axis interferes with the measurement data of the sensors. In addition to this the use of the three different sensors makes the procedure more expensive.

USA patent publication document number U.S. Pat. No. 6,122,538A presents a device adapted for determining the position and orientation of a mobile medical imaging device. Here two different types of sensor are located on the device. The one type of sensor measures the angular direction of the device, while the other type of sensor measures at least one translational position of the device as compared to an external reference point. A serious disadvantage of such systems is that they require an active external reference point that a proportion of the sensors can use as a reference to determine the position or direction of the device.

Similarly, other systems use an active external reference point, for example, the publication of Tiesenhausen et al., “A new mobile and light-weight navigation system for interventional radiology,” International Congress and Exhibition “Computer Assisted Radiology and Surgery” (CARS), International Congress Series 1281, pages 412-417 (2005) presents a system in which a navigation camera and trackers are used while connected to a portable computer, which also operates as a display and as the navigation system required for the positioning of the medical device. The disadvantage of this system is that the navigation camera and the trackers must be in clear sight of each other at all times, which significantly restricts the movement of the personnel and the possible arrangement of the equipment in the room.

A biopsy needle may also be positioned using electromagnetic irradiation, such as the solution presented in the publication of Kim et al., “CT-guided liver biopsy with electromagnetic tracking: results from a single-center prospective randomized controlled trial,” American Journal of Roentgenology, Vol. 203, No. 6, pages W715-W723 (2014). This is a system that uses electromagnetic tracking in the case of CT-guided biopsy. The disadvantage of the system is that it is complicated to set up, and occupies a great deal of space, especially the electromagnetic signal generator and the shielding apparatus. In addition it may cause interference with other medical devices operating nearby.

As a consequence of the above there is a need for a system with the use of which the direction of a medical device may be monitored and predicted in such a way that during use it is permitted for the medical device to rotate around its own axis and in the case of which the calibration of the device is simple.

### BRIEF DESCRIPTION OF THE INVENTION

The present invention is based on the recognition that in the case of the use of a sensing system the orientation of the medical device may be monitored in the course of biopsies and similar medical procedures if the medical device is positioned in several suitably selected, known orientations. Using these orientations the medical device is calibrated to the coordinate system of the diagnostic imaging device (e.g. CT scanner) as reference, and the direction of the needle, i.e. of the puncturing is determined as compared to the orientation of the measuring device (hereinafter effective direction). In this way there is no need for additional external active devices in order to be able to monitor the orientation and effective direction of the medical instrument.

In accordance with the above the present invention relates to a system that contains a medical device, a measuring device, a calibrating device and an external device that communicates with the measuring device, where the external device contains a communication module, a control unit, a program, a power source and display, and it is characteristic of the system that the measuring device is connected to the medical device, which measuring device contains a sensing system, which is preferably a gyroscope and accelerometer, a power source, a communication module and control unit, and the calibrating device has at least one position adapted for calibrating the medical device.

According to a preferred embodiment of the system according to the invention the calibrating device has at least two positions adapted for calibrating the medical device.

According to a preferred embodiment of the system according to the invention the calibrating device has at least three positions adapted for calibrating the medical device.

According to a preferred embodiment of the system according to the invention the calibrating device uses at least one rotation matrix.

According to a preferred embodiment of the system according to the invention it contains an imaging apparatus for monitoring the position of the medical device in the body.

According to a preferred embodiment of the system according to the invention the medical device is a biopsy needle, which has a needle.

According to a preferred embodiment of the system according to the invention the medical device is an ablation needle, which has a needle.

According to a preferred embodiment of the system according to the invention the medical device is a drainage needle, which has a needle.

According to a preferred embodiment of the system according to the invention the medical device also has a connection element.

The present invention also relates to the use of the system for monitoring the orientation of medical devices.

In the figures

FIG. 1 shows a front view of a preferred embodiment of the medical device according to the invention with the measuring device;

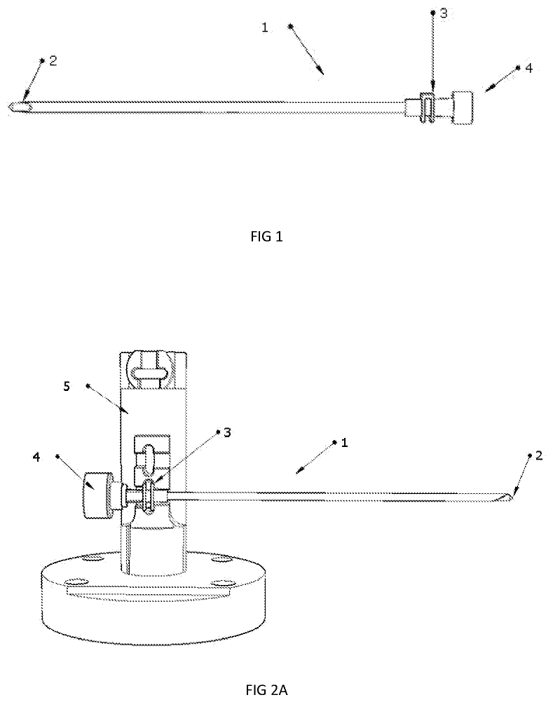

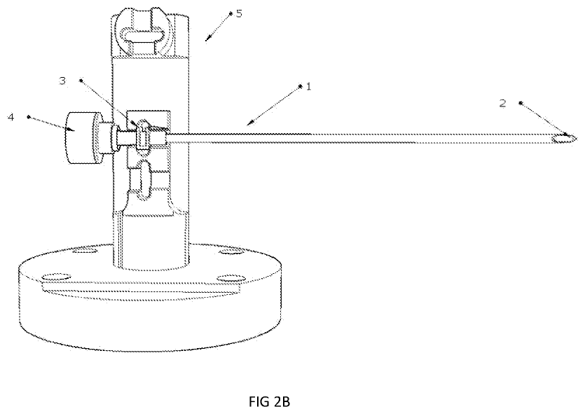

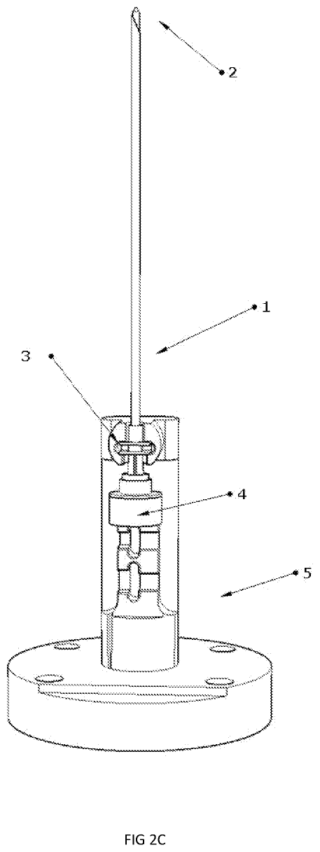

FIG. 2a shows a front view of the medical device placed in the first position of the calibrating device according to the invention;

FIG. 2b shows a front view of the medical device placed in the second position of the calibrating device according to the invention;

FIG. 2c shows a front view of the medical device placed in the third position of the calibrating device according to the invention;

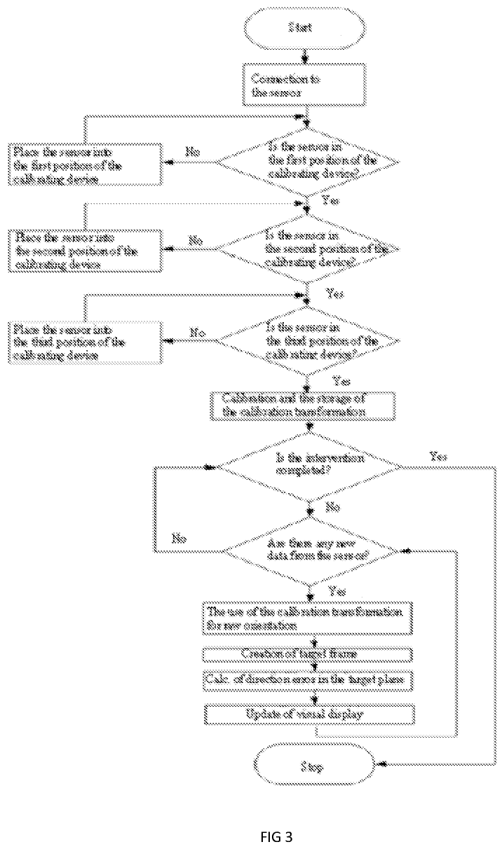

FIG. 3 shows a block diagram of the software of the system according to the invention;

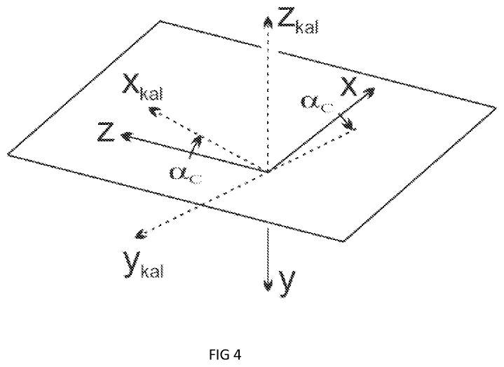

FIG. 4 shows the relationship between the world frame and the frame of the calibrating device.

### DETAILED DESCRIPTION OF THE INVENTION

The essence of the system according to the invention is that the medical device contains a measuring device, which measuring device is a MEMS IMU sensor containing at least one gyroscope and one accelerometer and during calibration it is placed in positions of known orientation in an appropriately selected world coordinate system.

The present invention provides a system with which biopsies and similar medical procedures may be performed in the bodies of humans or animals.

In the context of the present invention a MEMS IMU sensor is understood to mean those micro-electro-mechanical systems (MEMS) that contain an accelerometer and/or gyroscope, furthermore, optionally they may also contain a magnetometer and other sensors.

In the context of the present invention orientation is understood to mean geometric orientation.

In the context of the present invention effective direction is understood to mean the direction of the medical device, and, as a result, of the puncturing as compared to the orientation of the measuring device.

In the context of the present invention skin point is understood to mean the position where the medical device punctures into the patient's body in order to reach the target. Depending on the resolution of the imaging device used and clinical aspects the skin point may be typically located within a sphere with a radius of 1 mm.

Within the scope of the present invention target is understood to mean that part of space located in the human or animal body that may be described with coordinates in 3D space where the medical device is to be taken to. Characteristically this target is a lesion, which is injured tissue or tissue that has been subjected to an abnormal change. The lesion may occur both in humans and in animals. The target may also be a given part of tissue that is to be examined.

In the context of the present invention target area is understood to mean a space part delimited by a spherical surface that either partially or entirely contains the tissue structure from which the doctor wishes to take a sample or which the doctor wishes to ablate or drain fluid from. The target area is characterised by the coordinates of the centre point of the target area and the radius of the sphere. The identification of the target area takes place with respect to the skin point.

The medical device depicted in FIG. 1 marked in its entirety with reference sign 1 consists of a needle 2 and a connection element 3, through which a measuring device 4 is connected to the medical device 1.

The system also contains a calibrating device 5 (FIG. 2a), which calibrating device 5 serves for securing the medical device 1 in given (known) positions. By using the calibrating device the medical device 1 may be secured on a CT table (not depicted) or on another solid object with known orientation as compared to the CT table for the duration of the calibration.

The structure of the calibrating device 5 is such so that it may be secured to flat surfaces with screws, clamps, rubber or other permanent or temporary securing devices. Furthermore, it has various positions (FIGS. 2a, 2b, 2c), in which the medical device 1 may be placed and secured even using clamps, or positions of such a design so that the medical device 1 may be clicked into these positions by exerting sufficient force, and removal from the calibrating device 5 also demands the exertion of force. Those possibilities are not excluded when the long medical device 1 may be pushed into a given opening of the calibrating device 5 so that it fits tightly up to the material of the calibrating device 5 and so that sufficient adhesive friction force is created between the materials of the medical device 1 and the calibrating device 5 so that they remain immovable in spite of the occurrence of weak forces (such as a small movement of the flat surface, personnel passing nearby the device, wind created by apparatuses).

An additional element of the system is an external device that is in a data transfer connection with the measuring device 4 and which, during the procedure, performs the necessary calculations and displays the result in a graphic, easily understandable form.

In other words during the use of the system the medical device 1 calibrated with the use of the calibration device 5 continuously sends signals about its orientation via the measuring device 4 to the external device, which external device calculates the effective direction of the medical device 1 from the initial parameters, the data obtained during calibration, and from the data sent by the measuring device 4 during the movement of the medical device 1. The external device preferably displays the given effective direction of the medical device 1 and its deviation from the target in graphic form, even more preferably it is displayed in the tissue environment displayed by the imaging apparatus, in this way any angle errors occurring may be easily corrected.

The measuring device 4 contains a sensor system. The task of the sensor system is to measure the orientation of the medical device 1. This may take place using various visual odometry devices or preferably by using a combination of gyroscope and accelerometer. However, no device or devices are excluded that are capable of, independently or in combination, measuring the orientation of the medical device 1 without the use of an external active reference.

Internal cameras are used during visual odometry, which survey the environment and periodically determine the movement of the medical device 1 from the changes to the images.

An accelerometer is a sensor that measure the force exerted on a mass on the basis of Newton's second law of motion. Usually this is performed by deriving force from the measurement of capacitance.

A gyroscope is a sensor with which angular velocity may be measured. A gyroscope operating on the classical principle contains a balanced mass supported on bearings, which is spun up to a high speed of rotation, and it is surrounded by a frame that enables its free movement. In this way, in the case the frame is moved, the gyroscope retains its original rotation plane and the change in the angle may be calculated from the movement of the frame. The operation principle of MEMS gyroscopes is similar, only instead of rotational movement harmonic vibration movement is used, so that angular velocity is calculated from the varying Coriolis force dependent on the direction of movement and its speed (C. Acar and A. Shkel, MEMS Vibratory Gyroscopes: Structural Approaches to Improve Robustness. Springer Science & Business Media, 2008; S. Beeby, MEMS Mechanical Sensors. Artech House, 2004). The gyroscope preferably used in the course of the invention operates on the capacitive principle and the magnitude detected by it is proportionate to the angular velocity.

The measuring device 4 also contains a processor, power source and a communication module.

The control unit performs the reading and processing of the data provided by the measuring device 4. Furthermore, the control unit stores the program required for the measurement, performs the necessary calculations, and forwards the data to the external unit via the communication module. In the context of the present invention control unit is understood to mean a microcontroller, or system on chip (SoC), furthermore all similar devices that perform calculations within information technology systems, store the data, and control the peripheries, such as the communication module.

A power source supplies the measuring device 4 with power. This is basically understood to mean a power source built into the measuring device 4, which may be chargeable and/or replaceable, but it is not excluded that a single-use power source is used, or that the measuring device 4 is connected to an external power source, such as an external battery (power bank), or that the mains electricity supply is used as the power supply.

The communication module enables communication with the external unit, or the flow of data between these, with this being performed in a wireless and/or wired way.

The measuring device 4 communicates with an external device via the communication module and sends the data processed by the control unit of the measuring device 4 to the control unit of the external device. In addition to this the external device also has a display and a program displayed on this, and the display can be used to monitor in this program the effective direction of the virtual medical device 1 created on the basis of the data of the measuring device 4 as compared to the target direction.

During its operation the effective direction of the medical device 1 provided with the calibrated measuring device 4 and the target may be monitored on the display of the external device. The angle deviation may be corrected on the basis of the display if the medical device 1 is not aimed in the direction of the target. The display preferably depicts the position of the virtual medical device 1 and also the target towards it is progressing in a graphic way. The possibility is not ruled out of the external device providing only textual information on whether the direction of the medical device 1 is correct and if it is not it provides information on the direction and magnitude of the correction required in order to reach the target. Naturally, a combination of the graphic and textual/numerical display modes is also conceivable.

The calibrating device 5 is shown in FIG. 2, it is secured on a CT table, and the medical device 1 is secured between the securing clamps of the calibrating device 5 via its connection element 3. This connection element 3 of the medical device 1 is not an essential part of the invention, it merely facilitates the positioning of the medical device 1 into the calibration device 5 and its securing there in the case of certain types of medical device 1.

The algorithm of the software operating the system is shown in the block diagram depicted in FIG. 3. As the first step the measuring device 4 is connected to the external device via the communication module. In the next step the medical device 1 provided with the measuring device 4 is placed into the calibrating device 5 for the purpose of performing the calibration. In order for the program running on the external device to be easy to use and user-friendly it may be preferably supplied with a checking step to determine whether the medical device 1 has been placed in the calibrating device 5 for the calibration.

The purpose of the calibration is to determine the reference frame of the measuring device 4 in a coordinate system corresponding to a predetermined world frame (world frame is preferably understood to mean the coordinate system of an imaging device, such as a CT scanner), i.e. the mathematical entity required in order to correlate the two frames with each other. Mathematical entity is understood to mean quaternion, Euler vector, etc., preferably a rotation matrix. The calculations related to the mathematical entities are presented in the following citation: L. M. Surhone, M. T. Timpledon, and S. F. Marseken, Rotation Representation (mathematics): Rotation Matrix, Axis Angle, Euler Angles, Quaternions and Spatial Rotation. Betascript Publishing, 2010. Furthermore, the determination of the direction of the needle 2 is also an objective in the frame of the measuring device 4. The calibrating device 5 is placed on the CT table or on another apparatus determining a world frame.

Calibration of various complexity may be required depending on the features of the technologies used.

If the vertical axis of the frame of the calibrating device 5 and the frame of the measuring device 4 coincide, the medical device 1 provided with the measuring device 4 is placed in the calibrating device 5 in a direction (not vertical) known in the world frame. In the opposite case the medical device 1 is placed into the securing seats oriented in two different directions one after the other.

If the precise direction of the needle 2 in the measuring device 4 frame is not known, the medical device 1 is secured in a further position by rotating it around the axis of the needle 2 in the calibrating device 5.

After the calibration has been performed the measurement data produced during the measurement performed by the measuring device 4 of the given orientation of the medical device 1 can be placed in the world frame using the data obtained from the calibration and the change of the effective direction of the medical device 1 may be monitored.

The measuring device 4 provides measurement data on the orientation of the measuring device 4 in every new orientation of the medical device 1 while it is being moved. Furthermore, after performing the error calculation the program compares the direction of the measuring device 4 and the position of the target in the world frame. In this way, on the graphic interface, it is easy to depict how to change the direction of the medical device 1 if it is not progressing towards the target.

In the course of the use of the system, after the target has been reached, the imaging apparatus (not depicted) is used to check whether the medical device 1 is actually in the right position. If it is, then the sample may be taken or the other types of desired intervention may be performed. Naturally, the possibility is not ruled out when the system is used without checking being performed with the imaging apparatus. Moreover, on the basis of the images supplied by the imaging apparatus the pathway of the medical device 1 leading towards the target may even be monitored in the period before the target is reached.

During the communication between the external device and the measuring device 4 the measuring device 4 sends the orientation data to the external device, which external device then performs the calibration, the geometric transformations, and the visual display with the use of these data, in addition it sends control signals to the measuring device 4 with which it restarts, stops and starts, etc. the measuring device 4.

The system according to the present invention may be used in procedures using CT-guided targeting (biopsy, aspiration, drainage, ablation), in the case of procedures using targeting guided by other imaging apparatus (ultrasound, MRI, X-ray), and in the case of other interventions, for example, orthopaedic, neurosurgery and robotic surgery interventions.

The medical device 1 according to the present invention is understood to mean the devices used in the case of these procedures, with which a target that cannot be seen with the naked eye has to be reached (biopsy needle, ablation needle, drainage needle, etc.).

### EXAMPLES

### Example 1: Single-Step Calibration

A right-handed, right-angled coordinate system is used for the calculations on the CT table, on which the calibration is also performed; this will be the world frame. The X-Z axes are placed on the horizontal plane in such a way that the medical device 1 placed in the calibrating device 5 points in the direction of the Z-axis of the world frame.

The deviation between the world frame and the frame of the calibrating device 5 can be seen in FIG. 4, which is marked with the angle α_{c}. The angle α_{c} depends on the position of the calibrating device 5, and if it is unknown to the person skilled in the art, its value may be measured in a simple way. The value of the rotation matrix belonging to the frame of the calibrating device 5 and the world frame may be determined on the basis of the following formula in the knowledge of α_{c}:

$${R}_{ref 1} = [ \begin{matrix}sin ( {a}_{C} ) & - cos ( {a}_{C} ) & 0 \\ 0 & 0 & - 1 \\ cos ( {a}_{C} ) & sin ( {a}_{C} ) & 0\end{matrix} ] .$$

In the case of single-step calibration the medical device 1 provided with the measuring device 4 is secured in the calibration device 5 in a position parallel to the top of the CT table. In its secured position the direction of the needle 2 as compared to the measuring device 4 is known, because the positions of the sensors in the measuring device 4 are known precisely. Furthermore, the calibrating device 5 and the vertical axis of measuring device 4 reference coincide, therefore it is sufficient to perform the calibration in the horizontal direction.

Following the calibration the axis directions of the calibrating device 5 as compared to the reference frames of the measuring device 4 will be known, therefore the inverse of the rotation matrix formed from these will provide the value of the matrix R_{ref2}. As a result the orientation of the measuring device 4 as compared to the world frame may be calculated in the following way:

R(t)=R_{ref}R_{measured}(t),
where R_{ref}=R_{ref1}R_{ref2},
and with this the direction of the needle 2 may be calculated in the form R(t)*i.

### Example 2: Three-Step Calibration

The single-step calibration according to example 1 is performed in the same way with the difference that the direction of the needle 2 is not known and the frame of the calibrating device 5 and the vertical axis of the reference frame of the measuring device 4 do not coincide. This occurs in cases when the precise orientation of the various sensors placed in the measuring device 4 and the relative orientation of the medical device 1 are not precisely known. Therefore, following the first calibration step it is also necessary to determine the direction of the needle 2, for which, by rotating the medical device 1 around the axis of the needle 2, it is secured in the calibrating device 5 in a second calibration position. The direction of the needle 2 may be calculated on the basis of the closer interpretation of the axis of rotation of the difference of the rotations (dR=R₁⁻¹R₂) to the nominal direction.

Additionally, a third calibration position has to be set in order to determine the difference of the vertical axes, in which the direction of the needle 2 is in the horizontal plane, or in the plane between the previous position and the vertical direction. The vertical direction as compared to the reference value of the measuring device 4 may be calculated from this. From this point onwards, the calculation is continued using the rotation matrix R_{ref2} obtained from the calibration steps in the way according to example 1.

### Example 3: The Use of the System During a Biopsy

The device according to the invention is used in the case of a CT-guided lung biopsy by following the following steps:
The doctor places a reference X-ray absorbing grid onto the chest of the lying patient.
Following this a low-dose scan is performed of the examined region (lung) using a Siemens Definition Edge CT apparatus while the patient is lying on the CT table.
The ideal needle path and the skin point and angle pair required for this are determined in the external unit, which is a computer, on the basis of the axial images, the multidirectional (even any optional direction) reconstructions and the reference grid.
The doctor marks the skin point on the patient's skin.
The assistant removes the calibrating device 5 from the sterile packet and places it on the CT table, which is essentially a securing seat for securing the medical device 1 in various positions with the positions detailed in example 2.
The assistant removes the medical device 1 used for the biopsy from the packet, which is a coaxial biopsy needle, and fits the measuring device onto it. Following this the assistant switches on the measuring device 4 and places the medical device 1 into the calibrating device 5 and calibrates it.
In the measuring device 4 there is a MEMS IMU 9250 sensor provided with an accelerometer and a gyroscope, a power source, a communication unit using the Bluetooth standard and a microcontroller.
The assistant sterilises and anaesthetises the patient's skin at the skin point. Following this the assistant sets the measured angle pair in the program running on the computer.
In the following step the doctor removes the medical device 1 along with the measuring device 4 from the calibrating device 5 and places it on the skin point. In addition the doctor sets the position of the medical device 1 so it stands in the desired initial position in the graphic interface (in other words the crosshairs indicating the direction of the medical device 1 and the tumour targeted with the biopsy overlap each other) and so that the needle path error is minimal.
Maintaining the angle indicated by the external unit the doctor inserts the medical device 1 into the patient's body to the depth depending on the situation. The determination of the depth belongs to the mandatory knowledge of the person skilled in the art, among other aspects, depending on the sensitivity of the patient, the characteristics of the given body part, and the thickness of the layer of fat on the patient's body.
Following this the doctor leaves the room containing the CT apparatus and scans the patient in order to check the position and direction of the medical device 1.
In the following step the doctor examines the images to determine whether the direction of the medical device 1 is correct and the distance the medical device 1 still has to move in order to reach the tumour.
In the knowledge of this information the doctor once again enters the CT room and pushes the medical device 1 in the patient's body up to the tumour.
Then the doctor leaves the CT room and uses the CT apparatus to check whether the medical device 1 has punctured the tumour.
If it has, the doctor removes the measuring device 4 from the medical device 1 and pulls out the internal needle, thereby leaving a channel through which the doctor introduces a semiautomatic biopsy gun and takes a sample from the tumour.
If the sample is good, the channel is removed and the wound is dressed, and then for the purpose of checking for complications a low-dose control chest scan is performed.

An advantage of the invention is that there is no need for the use of an active external reference in order to use the system, only preliminary calibration is required.

A further advantage of the invention is that the use of the system is fast, the built environment and metal objects do not interfere with the precision of the measurement.

A further advantage of the invention is that the measuring device 4 only has to have an accelerometer and a gyroscope; additional measuring instruments are not required for its use.

A further advantage of the invention is that the small size and mass of the measuring instrument do not have a negative effect on the performance of the intervention.

A further advantage of the invention is that it supplements the existing devices used in medical practice, therefore there is no need for the introduction of complete, new devices in order to use the invention.

A further advantage of the invention is that its use does not require the operation of complex software systems using multiple parameters, because the external device only requires the adjustment of those parameters that the doctor otherwise takes into consideration during the intervention.

1. System for monitoring the orientation of medical devices (1), which contains a medical device (1), a measuring device (4), a calibrating device (5) and an external device that communicates with the measuring device (4), which external device contains a communication module, a control unit, a program, a power source and display, characterised by that the measuring device (4) is connected to the medical device (1), which measuring device (4) contains a sensing system, which is preferably a gyroscope and accelerometer, a power source, a communication module and control unit, and the calibrating device (5) has at least one position adapted for calibrating the medical device (1).

2. System according to claim 1, characterised by that the calibrating device (5) has at least two positions adapted for calibrating the medical device (1).

3. System according to claim 1, characterised by that the calibrating device (5) has at least three positions adapted for calibrating the medical device (1).

4. System according to claim 1, characterised by that the calibrating device (5) uses at least one rotation matrix.

5. System according to claim 1, characterised by that it contains an imaging apparatus for monitoring the position of the medical device (1) in the body.

6. System according to claim 1, characterised by that the medical device (1) is a biopsy needle, which has a needle (2).

7. System according to claim 1, characterised by that the medical device (1) is an ablation needle, which has a needle (2).

8. System according to claim 1, characterised by that the medical device (1) is a drainage needle, which has a needle (2).

9. System according to claim 1, characterised by that the medical device (1) also has a connection element (3).

10. A method for monitoring the orientation of medical devices (1), comprising monitoring by the system according to claim 1.
